(19) United States
(12) Patent Application Publication
(10) Pub. No.: US 2025/0248779 A1
(43) Pub. Date: Aug. 7, 2025

(54) Container with Improved Medical Device Nest Support and Deformation Resistance

(71) Applicant: Becton, Dickinson and Company, Franklin Lakes, NJ (US)

(72) Inventors: Simon Rempfer, St Hilaire de la Cote (FR); Anaïs Eymery, Saint-Georges-de-Commiers (FR); Ferdinand Lavigne, Seyssinet-Pariset (FR); Florian Pineda, Grenoble (FR); Laurent Jacquier, Saint Cassien (FR); Aurélie Barcelo, Saint-Martin-d'Heres (FR)

(21) Appl. No.: 18/432,184

(22) Filed: Feb. 5, 2024

(51) Int. Cl. A61B 50/26; A61B 50/00; A61B 50/30

(52) U.S. Cl. CPC A61B 50/26; A61B 50/30; A61B 2050/005

(57) ABSTRACT

Provided herein is a tub for holding one or more nests of medical components. The tub includes a bottom wall, a plurality of sidewalls extending upwardly from the bottom wall, and a top flange formed about a top edge of the plurality of sidewalls. Each sidewall is configured as a stepped wall including a lower wall portion and an upper wall portion, with a horizontal ledge formed between the lower wall portion and the upper wall portion that is in parallel with the bottom wall. The tub also includes one or more support members extending up from the bottom wall, with each of the one or more support members having a top surface that is vertically aligned with or vertically below the ledges of the plurality of sidewalls, so as to be positioned to support a bottommost nest of the one or more nests when placed within the tub.

### BACKGROUND OF THE INVENTION

### Field of the Invention

The present disclosure relates generally to nest arrangements for the packaging of medical containers (e.g., syringes) and/or components thereof (e.g., stoppers) and, more particularly, to an associated container, or tub, in which such nest arrangements are received.

### Description of Related Art

Medical containers and components for use with such containers, such as pre-fillable or prefilled syringes, cartridges, barrels, or stoppers, often need to be transported from one site to another site during or after manufacturing. For instance, medical containers or components thereof may be transported from a manufacturing site to a site at which the medical containers may be filled with a medicinal fluid. As used herein, a “medical fluid” can refer to a medication or another therapeutic agent used for treatment of chronic or acute conditions, as are known in the art. Exemplary therapeutic agents can include, for example, drugs, chemicals, biological, or biochemical substances that, when delivered in a therapeutically effective amount to the patient, achieve a desired therapeutic effect.

During transportation, the medical containers or components may be supported by a holding device, such as a tray or “nest.” As known in the art, nests are available for holding/transporting syringe barrels or stoppers to be inserted in such syringe barrels (such as after pre-filling of a syringe barrel). One or more nests may be disposed within a holding and transport container, which may be referred to as a tub, with the tub having an opening allowing for placement of one or a plurality of nests therein and subsequent sealing thereof by a sealing cover.

With regard to tubs that are used for transporting a plurality of nests (and the medical containers or components held thereby), current tub designs have numerous potential drawbacks regarding the supporting of nests held therein and/or regarding the removal of nests therefrom. In particular, for tubs designed to hold nests that retain syringe stoppers therein, the particular construction of existing tubs is undesirable for numerous reasons.

As one example, for nests configured to retain syringe stoppers therein, the tubs used to hold such nests may not provide adequate support to the nests during storage/shipment. Tubs for holding nests of syringe stoppers may often include sidewalls having a stepped construction—with the sidewalls having a horizontally-oriented ledge or support surface formed thereon located between a bottom wall and top opening of the tub, with the ledge or support surface supporting the nests positioned within the tub. That is, the ledge or support surface formed on the internal surface of the sidewalls supports a perimeter of a support plate of the bottom-most nest held in the tub. However, due to design restrictions and the desire to accommodate nests of various designs (e.g., nests with 100 and 160 chimneys), the width of the ledge or support surface must be limited, such that only a very narrow support surface is provided on which a support plate of the nest(s) may rest. This narrow ledge or support surface may lead to an increased deflection of the bottom-most nest, which can lead to issues on the customer side regarding the insertion/removal of stoppers from the chimneys of the nest.

As another example, for nests configured to retain syringe stoppers therein, the tubs used to hold such nests are prone to tub deformation after prolonged periods of use. That is, as compared to tubs designed to hold nests of syringe barrels, tubs that hold nests of stoppers retain the nests on ledges formed much lower on the sidewalls of the tub. This general design results in a tub having sidewalls of increased height that are more prone to deflect over time (i.e., a “box effect”) due to the tub being formed as an injection molded component that may undergo stress relaxation after prolonged periods of time. A deflection of the sidewalls of the tub resulting from such stress relaxation may inhibit removal of the nest(s) from the tub (e.g., using an automatic unloading machine), as such deflection may result in contact/friction between the nest(s) and the tub as the nest are being removed.

Accordingly, a need exists in the art for a tub design (for nests carrying syringe stoppers) that provides additional support for the nest retained therein, so as to prevent unwanted deflection of the nests. A need also exists in the art for a tub design (for nests carrying syringe stoppers) that is more resistive to sidewall deflection and deformation that might result from stress relaxation that occurs in the sidewalls after prolonged periods of time, so as to prevent potential issues with removing the nests from the tub that might result from such deflection and deformation.

### SUMMARY OF THE INVENTION

Provided herein is a tub for holding one or more nests each including a plurality of chimneys configured to retain medical components therein. The tube includes a bottom wall and a plurality of sidewalls extending upwardly from the bottom wall and including an inner wall surface and an outer wall surface, wherein each of the plurality of sidewalls comprises a stepped wall including a lower wall portion and an upper wall portion, with a ledge formed between the lower wall portion and the upper wall portion, the ledge comprising a horizontal ledge in parallel with the bottom wall. The tub also includes a top flange formed about a top edge of the plurality of sidewalls and one or more support members extending up from the bottom wall, each of the one or more support members having a top surface that is vertically aligned with or vertically below the ledges of the plurality of sidewalls, with the one or more support members positioned to support a bottommost nest of the one or more nests when placed within the tub.

In accordance with some aspects of the disclosure, the one or more support members comprise a plurality of steps formed as part of the plurality of sidewalls, each of the plurality of steps extending inwardly from a respective lower wall portion and configured to fit between adjacent chimneys of the bottommost nest, with the top surface of the each of the plurality of steps vertically aligned with the ledges of the plurality of sidewalls so as to contact a bottom surface of a support plate of the bottommost nest when placed within the tub.

In accordance with some aspects of the disclosure, the one or more support members comprise a plurality of ribs formed as part of the plurality of sidewalls, each of the plurality of ribs extending inwardly from a respective lower wall portion and configured to fit between adjacent chimneys of the bottommost nest, with the top surface of the each of the plurality of ribs vertically aligned with the ledges of the plurality of sidewalls so as to contact a bottom surface of a support plate of the bottommost nest when placed within the tub.

In accordance with some aspects of the disclosure, the one or more support members comprise one or more support columns extending up from the bottom wall, with the top surface of each of the one or more support columns positioned vertically below the ledges of the plurality of sidewalls, so as to support a lower edge of the plurality of chimneys of the bottommost nest when placed within the tub.

In accordance with some aspects of the disclosure, the one or more support columns comprise a first support column positioned adjacent a first sidewall of the plurality of sidewalls and a second support column positioned adjacent a second sidewall of the plurality of sidewalls opposite the first sidewall.

In accordance with some aspects of the disclosure, the one or more support columns comprise a single support column positioned so as to be centered on the bottom wall, the single support column covering a majority of the bottom wall.

In accordance with some aspects of the disclosure, the one or more support columns comprise a first pair of cylindrical support columns spaced apart from a first sidewall of the plurality of sidewalls and a second pair of cylindrical support columns spaced apart from a second sidewall of the plurality of sidewalls opposite the first sidewall.

In accordance with some aspects of the disclosure, the ledge extends outwardly from a top edge of the lower wall portion to a bottom edge of the upper wall portion, and wherein the ledge is configured to support a support plate of the bottom-most nest of the one or more nests held in the tub.

In accordance with some aspects of the disclosure, each of the plurality of sidewalls includes one or more guide features thereon extending upwardly from the ledge, with each of the one or more guide features defining a projection on the inner wall surface and a recess on the outer wall surface.

Also provided herein is a packaging system for storing a plurality of medical components. The packaging system includes one or more nests each configured to retain a plurality of medical components, with each of the one or more nests comprising a support plate having a top surface and a bottom surface and a plurality of chimneys formed on the support plate and configured to retain the plurality of medical components therein, each of the plurality of chimneys defining an opening in the support plate and comprising a cylindrical wall extending down from the bottom surface of the support plate, the cylindrical wall having a top edge joined to the support plate and a bottom edge. The packaging system also includes a tub comprising a bottom wall and a plurality of sidewalls extending upwardly from the bottom wall and including an inner wall surface and an outer wall surface, wherein each of the plurality of sidewalls comprises a stepped wall including a lower wall portion and an upper wall portion, with a ledge formed between the lower wall portion and the upper wall portion, the ledge comprising a horizontal ledge in parallel with the bottom wall. The tub further comprises a top flange formed about a top edge of the plurality of sidewalls and one or more support members extending up from the bottom wall, each of the one or more support members having a top surface that is vertically aligned with or vertically below the ledges of the plurality of sidewalls, with the one or more support members positioned to support a bottommost nest of the one or more nests when placed within the tub. The packaging system further includes a sealing cover applied over the tub and joined to the top flange.

In accordance with some aspects of the disclosure, the top surface of each of the one or more support members is vertically aligned with the ledges of the plurality of sidewalls, with the one or more support members configured to fit between adjacent chimneys of the bottommost nest and contact the bottom surface of the support plate of the bottommost nest when placed within the tub.

In accordance with some aspects of the disclosure, the top surface of each of the one or more support members is positioned vertically below the ledges of the plurality of sidewalls, with the one or more support members configured to contact the bottom edges of the plurality of chimneys of the bottommost nest when placed within the tub.

Also provided herein is a tub for holding one or more nests each configured to retain a plurality of medical components. The tub includes a bottom wall and a plurality of sidewalls extending upwardly from the bottom wall and including an inner wall surface and an outer wall surface, wherein each of the plurality of sidewalls comprises a stepped wall including a lower wall portion and an upper wall portion, with a horizontal oriented nest ledge formed between the lower wall portion and the upper wall portion, the nest ledge configured to support a bottom-most nest of the one or more nests. The tub also includes a top flange formed about a top edge of the plurality of sidewalls, with the top flange defining an upper opening of the tub. The upper wall portion on each of the plurality of sidewalls comprises a stiffening belt formed on the inner wall surface thereof, the stiffening belt configured to resist an inward bowing of the respective sidewall.

In accordance with some aspects of the disclosure, the upper wall portion comprises a stepped wall including a first portion and a second portion, and wherein the stiffening belt comprises a stiffening ledge formed between the first portion and the second portion.

In accordance with some aspects of the disclosure, the stiffening ledge on each respective sidewall of the plurality of sidewalls comprises a tapered ledge having a variable width along a length of the respective sidewall.

In accordance with some aspects of the disclosure, each of the plurality of sidewalls includes one or more guide features thereon extending upwardly from the nest ledge, with each of the one or more guide features defining a projection on the inner wall surface and a recess on the outer wall surface, and wherein the one or more guide features on each first of the plurality of sidewalls intersects the stiffening belt formed thereon.

Also provided herein is a tub for holding one or more nests each configured to retain a plurality of medical components. The tub includes a bottom wall and a plurality of sidewalls extending upwardly from the bottom wall and including an inner wall surface and an outer wall surface, wherein each of the plurality of sidewalls comprises a stepped wall including a lower wall portion and an upper wall portion, with a horizontal oriented nest ledge formed between the lower wall portion and the upper wall portion, the nest ledge configured to support a bottom-most nest of the one or more nests. The tub also includes a top flange formed about a top edge of the plurality of sidewalls, with the top flange defining an upper opening of the tub. The top flange comprises a vertically oriented lip formed about the outer edge thereof and extending downwardly therefrom, back toward the bottom wall, wherein the top flange and vertically oriented lip increase a rigidity of the plurality of sidewalls, such that the plurality of sidewalls are resistant to an inward bowing thereof.

Also provided herein is a tub for holding one or more nests each configured to retain a plurality of medical components. The tub includes a bottom wall and a plurality of sidewalls extending upwardly from the bottom wall and including an inner wall surface and an outer wall surface, wherein each of the plurality of sidewalls comprises a stepped wall including a lower wall portion and an upper wall portion, with a horizontal oriented nest ledge formed between the lower wall portion and the upper wall portion, the nest ledge configured to support a bottom-most nest of the one or more nests. The tub also includes a top flange formed about a top edge of the plurality of sidewalls, with the top flange defining an upper opening of the tub. A thickness of each of the plurality of sidewalls is greater than a thickness of the bottom wall, such that the plurality of sidewalls resist an inward bowing.

In accordance with some aspects of the disclosure, each of the plurality of sidewalls includes one or more guide features thereon extending upwardly from the nest ledge, with each of the one or more guide features defining a projection on the inner wall surface and a recess on the outer wall surface, and wherein the thickness of each of the plurality of sidewalls is increased at locations of the projections of the guide features, at locations other than the projections of the guide features, or at both the locations of the projections of the guide features and locations other than the projections of the guide features, such that the plurality of sidewalls may have a varied thickness or a constant thickness.

In accordance with some aspects of the disclosure, a thickness of the top flange is greater than a thickness of the bottom wall and equal to the thickness of each of the plurality of sidewalls.

### BRIEF DESCRIPTION OF THE DRAWINGS

FIG. 1 is a perspective view of a packaging system for storing and shipping a plurality of medical components, according to an embodiment of the disclosure;

FIG. 2 is an exploded view of the packaging system of FIG. 1;

FIG. 3 is a top perspective view of a tub that may be included in the packaging system of FIG. 1, according to an embodiment of the disclosure;

FIG. 4 is a bottom perspective view of the tub of FIG. 3;

FIG. 5 is a cross-sectional view taken along line 5-5 of FIG. 4, and with a bottom-most nest retained in the tub;

FIG. 6 is a top perspective view of a tub that may be included in the packaging system of FIG. 1, according to an embodiment of the disclosure;

FIG. 7 is a bottom perspective view of the tub of FIG. 6;

FIG. 8 is a top perspective view of a tub that may be included in the packaging system of FIG. 1, according to an embodiment of the disclosure;

FIG. 9 is a bottom perspective view of the tub of FIG. 8;

FIG. 10 is a top perspective view of a tub that may be included in the packaging system of FIG. 1, according to an embodiment of the disclosure;

FIG. 11 is a bottom perspective view of the tub of FIG. 10;

FIG. 12 is a top view of a tub that may be included in the packaging system of FIG. 1, according to an embodiment of the disclosure;

FIG. 13 is a detailed perspective view of a portion of the tub of FIG. 12;

FIG. 14 shows the portion of the tub of FIG. 13, with a bottom-most nest retained in the tub;

FIG. 15 is a top view of a tub that may be included in the packaging system of FIG. 1, according to an embodiment of the disclosure;

FIG. 16 is a detailed perspective view of a portion of the tub of FIG. 15;

FIG. 17 shows the portion of the tub of FIG. 16, with a bottom-most nest retained in the tub;

FIG. 18 is a top perspective view of a tub that may be included in the packaging system of FIG. 1, according to an embodiment of the disclosure;

FIG. 19 is a bottom perspective view of the tub of FIG. 18;

FIG. 20 is a top perspective view of a tub that may be included in the packaging system of FIG. 1, according to an embodiment of the disclosure;

FIG. 21 is a bottom perspective view of the tub of FIG. 20;

FIG. 22 is a top perspective view of a tub that may be included in the packaging system of FIG. 1, according to an embodiment of the disclosure;

FIG. 23 is a detailed perspective view of a portion of the tub of FIG. 22;

FIG. 24 is a top perspective view of a tub that may be included in the packaging system of FIG. 1, according to an embodiment of the disclosure;

FIG. 25 is a top perspective view of a tub that may be included in the packaging system of FIG. 1, according to an embodiment of the disclosure;

FIG. 26 is a top perspective view of a tub that may be included in the packaging system of FIG. 1, according to an embodiment of the disclosure; and

FIG. 27 is a top perspective view of a tub that may be included in the packaging system of FIG. 1, according to an embodiment of the disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

Aspects and embodiments of the disclosure are directed to tubs that are used for transporting a plurality of nests and the medical containers or components held thereby. The tubs are designed so as to provide for the nested stacking of empty tubs during shipping, while minimizing the possibility of tub deformation resulting from such nested stacking.

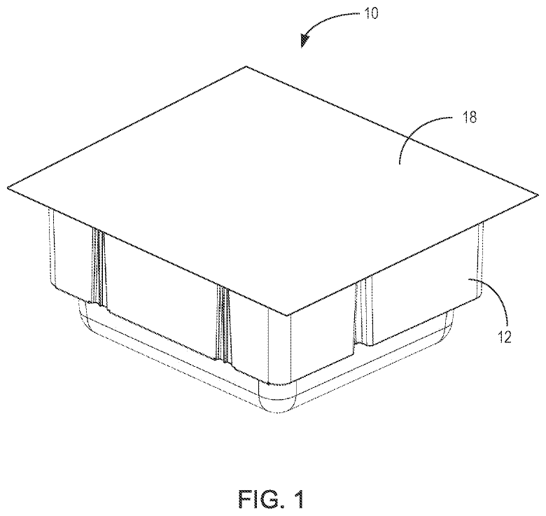

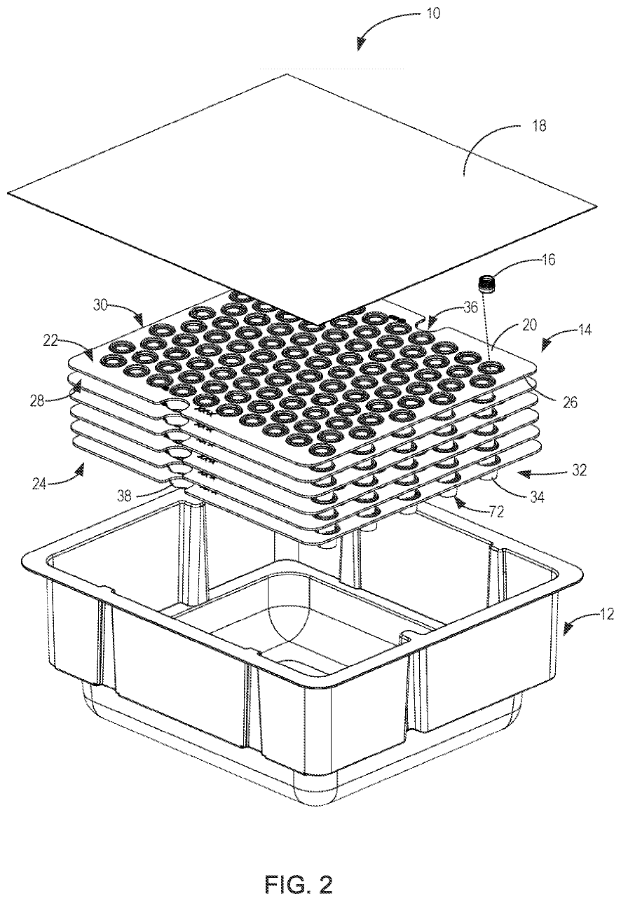

Referring to FIGS. 1 and 2, shown is a non-limiting embodiment of a packaging system 10 that may be used for the packaging and transporting of a plurality of medical components therein. The packaging system 10 includes a container or tub 12 in which a plurality of nests 14 may be housed, with each nest 14 retaining a plurality of medical components therein. In the illustrated embodiment, each of nests 14 is configured to retain therein a plurality of stoppers 16 that may be utilized in syringe containers (e.g., prefilled syringes), as known in the art, although it is recognized that nests 14 could alternatively retain other medical containers or components therein. The packaging system 10 also includes a sealing cover 18 that may be applied over the tub 12 after the nests 14 are packaged therein, to provide a sterile environment for storing and shipping the stoppers 16.

Exemplary nests 14 that may be retained in tub 12 are shown in FIG. 2. The nests 14 each comprise a support plate 20 having a first or upper surface 22, a second or lower surface 24, and a peripheral edge 26 extending about the support plate 20 between the upper surface 22 and the lower surface 24. In some examples, the perimeter of support plate 20 is substantially rectangular in shape-having longitudinal sides 28 and transverse sides 30. In some embodiments, the longitudinal sides 26 may be longer than the transverse sides 30. In other embodiments, the support plate 20 may be square, in which case the longitudinal sides 28 and the transverse sides 30 are the same length.

The nests 14 further comprise tubular walls, members, or ridges-referred to hereafter as “chimneys 32”—that protrude outward and downward from a plane defined by the upper surface 22 of the support plate 20. Each chimney 32 can include a cylindrical wall 34 that defines a receptacle configured to receive a stopper 16 therein, with the receptacle sized to retain an entirety of the stopper 16. The receptacle 36 includes a top opening at a top end of the chimney 32 and a bottom opening at a bottom end of the chimney 32. As shown in FIG. 2, according to some aspects of the disclosure, the nests 14 can be configured to include 100 chimneys 32 loaded with stoppers 16 for use with 1-3 mL syringes. Alternatively, the nests 14 can be configured to include 160 chimneys 32 loaded with stoppers 16 for use with 1 mlL syringes, or another number of chimneys 32 that is dependent on the size of the stoppers 16 retained therein.

In some embodiments, the nests 14 may further include therein median finger notches 36 on opposing longitudinal sides 28 (or transverse sides 30) of the plate 20, with each of the finger notches 36 including a delimiting wall 38. The finger notches 36 allow the insertion of a user's fingers therethrough, for catching the nest 14, and the delimiting walls 38 provide increased contact surfaces for these fingers.

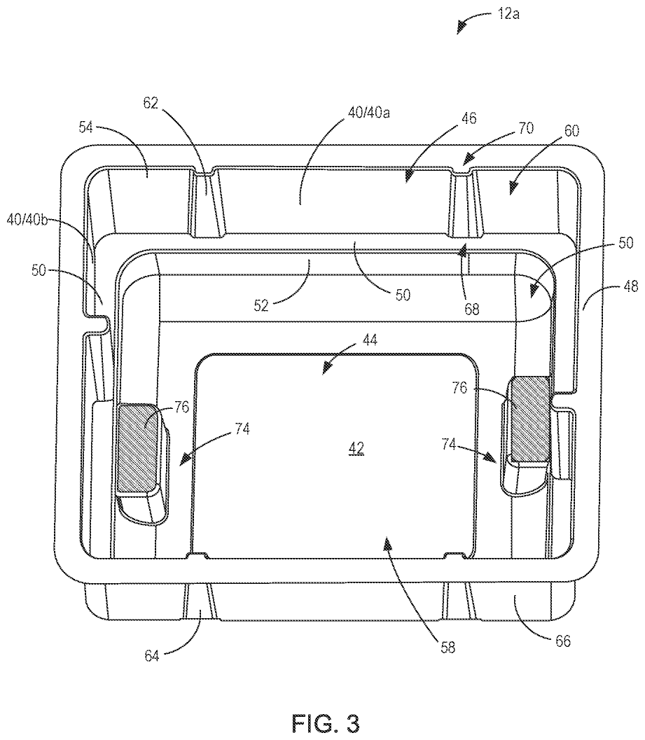

With reference now to FIGS. 3-5, a tub 12a that may be included in the packaging system 10 is shown and described in greater detail, in accordance with one embodiment of the disclosure. The tub 12a is formed by a single part of molded plastic material (e.g., an injection molded polystyrene tub) and comprises a plurality of sidewalls 40 that extend upwardly from a bottom wall 42 that defines a base of the tub 12a. Each of the sidewalls 40 may be configured as a sloped sidewall that does not extend exactly vertical from bottom wall 42, but instead extends up at an angle therefrom. The interior volume of the tub 12a is hollow and is defined by an interior surface 44 of the bottom wall 42 and an interior surface 46 of the sidewalls 40.

Opposite the bottom wall 42, the tub 12a is open such that the nests 12 may be inserted therein. A top flange 48 may be provided at a top end of the sidewalls 40 opposite to which the bottom wall 42 is located. The top flange 48 is configured to provide for coupling of sealing cover 18 (FIGS. 1 and 2) thereto (after one or more nests 12 having stoppers 14 contained therein are disposed in the tub 12a).

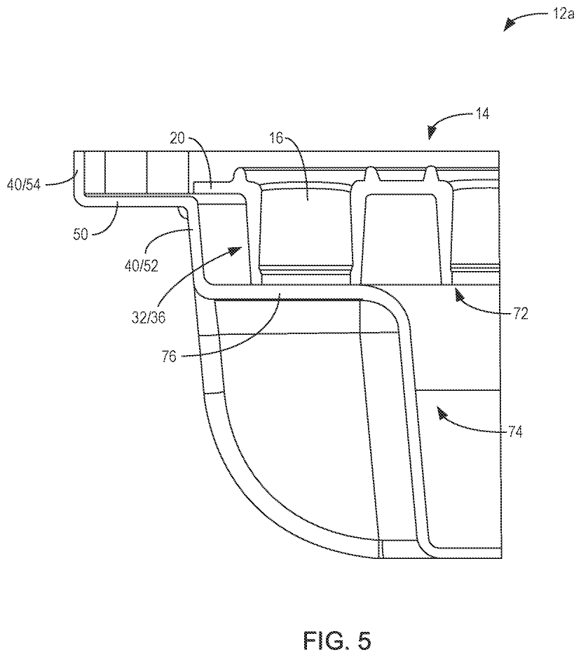

As illustrated in FIGS. 3-5, each of the sidewalls 40 may have a stepped construction that defines a (nest) ledge 50 positioned between a lower sidewall portion 52 and an upper sidewall portion 54 of each sidewall 40. The upper sidewall portion 54 is offset outwardly from the lower sidewall portion 52, such that an interior opening 56 defined by a perimeter of the ledge 50 has a smaller area than the opening 58 of the tub 12a defined by the flange 48. In use of the tub 12a, the ledge 50 provides a horizontal surface configured to support the plate 20 of a bottom-most nest 14 (FIG. 5) such that, when the plate 20 is disposed in the tub 12a, the lower surface 24 of the plate 20 is disposed on the ledge 50. While the ledge 50 is shown as extending continuously about a perimeter of the interior surface 46 of sidewalls 40, it is recognized that ledge 50 could also be formed discontinuously about a perimeter of the interior surface 46 of sidewalls 40.

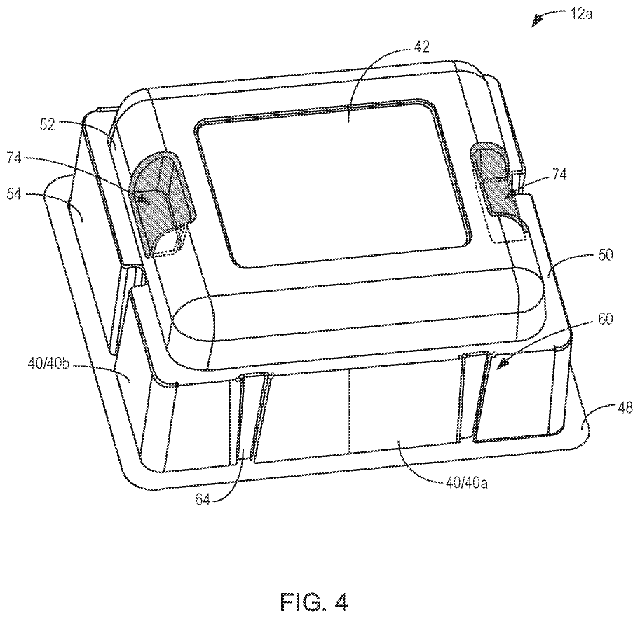

According to aspects of the disclosure, the tub 12a includes a plurality of features thereon that provide for stacking of the tub 12a with another tub of identical construction. In particular, the tub 12a includes features thereon that provide for the stacking of multiple tubs 12a when in an empty state (i.e., when not in use), such as when the tubs 12a are shipped from a facility where they are manufactured to a location where they are to be loaded with nests 14. As shown in FIGS. 3 and 4, at least some of the sidewalls 40 include guide features 60 thereon that provide for alignment and engaging of multiple tubs 12a when it is desired to stack tubs together. In the illustrated embodiment, each of first opposed lateral sidewalls 40a are shown as each including two guide features 60 thereon, with the guide features 60 aligned in identical positions on each of the lateral sidewalls 40a, while second opposed lateral sidewalls 40b are shown as each including a single guide feature, with the guide features 60 offset from one another on the opposing lateral sidewalls 40b. The guide features 60 are formed so as to appear as projections 62 that extend inwardly from the interior surface 46 of the sidewalls 40 and appear as recesses 64 depressed inwardly into an exterior surface 66 of the sidewalls 40. Each guide feature 60 extends vertically upwardly from a bottom end 68 thereof that is joined with ledge 50 to a top end 70 thereof that is vertically aligned with the top flange 48 (and opening 58) at the top end of the sidewalls 40.

According to aspects of the disclosure, the tub 12a also includes thereon one or more support members that provide additional support to the bottom-most nest 12 when the nest is placed within tub 12a. That is, it is recognized that the ledge 50 may provide only limited support to a bottom-most nest 14 retained within the tub 12a—this due to the chimneys 32 of the nest extending out almost to the edge of support plate 20 at least on two sides (i.e., transverse sides 30) of the support plate 20, such that there is little room for the support plate 20 to rest on ledges 50 along these sides. To address this lack of support provided by ledges 50, support members are provided in tub 12a that may engage the lower surface 24 of the support plate 20 or engage a bottom edge 72 of multiple chimneys 32, of the bottom-most nest 14. The additional support to bottom-most nest 14 provided by the support members may prevent a downward deflection or bending of the nest 14 that might occur if the nest is not properly supported in the tub 12a.

In the embodiment of FIGS. 3-5, support members are provided as a first support column 74 and a second support column 74 formed on or adjacent a pair of opposing sidewalls 40 of the tub 12a—i.e., on/adjacent the lower sidewall portion 52 of sidewalls 40. In the illustrated embodiment, the support columns 74 are formed on/adjacent the second opposed lateral sidewalls 40b where the transverse sides 30 sides of nest(s) 14 are positioned when placed within tub 12a—with it recognized that the ledges 50 provide less support to a bottom-most nest 14 along the transverse sides 30. Each of the support columns 74 may have a generally box-shaped profile, with the support columns 74 extending upwardly from the bottom wall 42 to a flat top surface 76. The height of the support columns 74 is such that the top surface 76 thereof is positioned vertically below the ledges 50 formed on sidewalls 40. With the support columns 74 configured as such, the top surfaces 76 of the support columns 74 are positioned so as to engage a bottom edge 72 of a plurality of chimneys 32 of the bottommost nest 14 when the nest is placed within the tub 12a and on the ledge 50 of the sidewalls 40. Each of the support columns 74 may be sized so as to make contact with a plurality of chimneys 32, such as between three (3) and five (5) chimneys 32 as a non-limiting example.

With the ledges 50 of tub 12a providing some support to the lower surface 24 of support plate 20 of the bottom-most nest 14 and the support columns 74 providing additional support to the underside of a plurality of chimneys 32 of the bottom-most nest 14, the nest is provided with multiple points/types of support within the tub 12a. With this support, the bottom-most nest 14 is prevented from deflecting/bending downward by an undesired amount, even when the bottom-most nest 14 is loaded with stoppers and additional nests are loaded on top of the bottom-most nest 14.

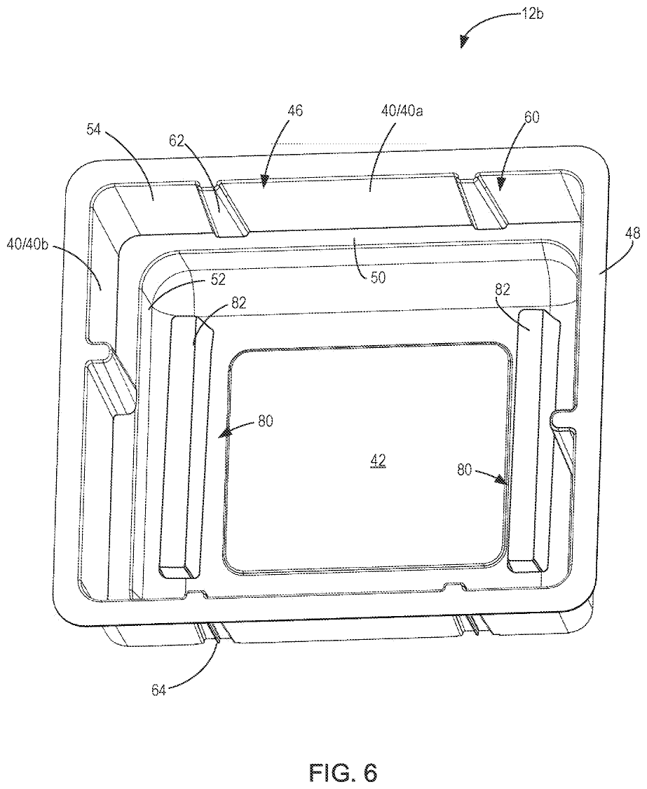

While FIGS. 3-5 show first and second support columns 74 of a specific size and at specific locations on tub 12a, it is recognized that the structure and/or number of support columns can vary according to other embodiments. FIGS. 6-11 illustrate additional embodiments of support columns that may be included in a tub to provide support to a bottom-most nest 14 loaded therein.

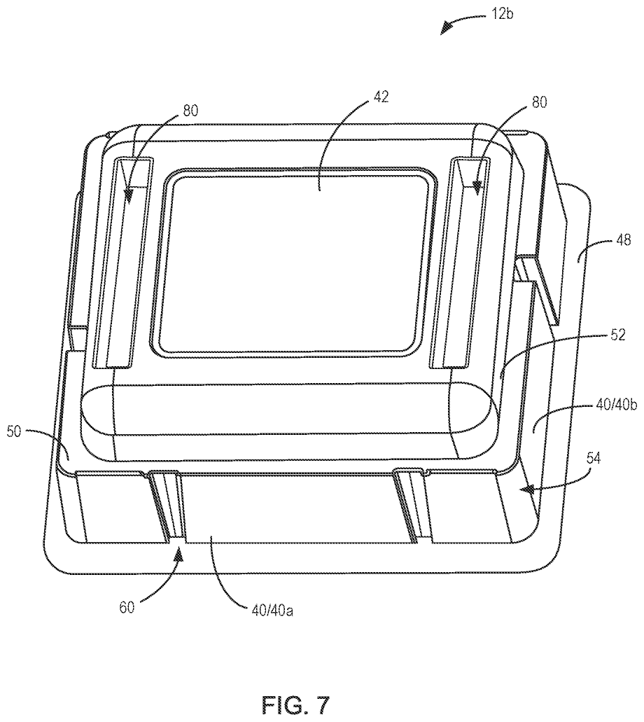

Referring to FIGS. 6 and 7, a tub 12b is shown that includes support members provided as a first support column 80 and a second support column 80 formed on or adjacent a pair of opposing sidewalls 40 of the tub—i.e., on/adjacent the lower sidewall portion 52. In the illustrated embodiment, the support columns 80 are formed on/adjacent the second opposed lateral sidewalls 40b where the transverse sides 30 of nest(s) 14 are positioned when placed within tub 12b—with it recognized that the ledges 50 provide less support to a bottom-most nest 14 along the transverse sides 30. Similar to the support columns 74 of FIGS. 3-5, each of the support columns 80 may have a generally box-shaped profile, with the support columns 80 extending upwardly from the bottom wall 42 to a flat top surface 82 and with a height of the support columns 80 positioning the top surface 82 vertically below the ledges 50 formed on sidewalls 40, such that the top surfaces 82 of the support columns 80 are positioned so as to engage a bottom edge 72 of a plurality of chimneys 32 (FIG. 2) of the bottommost nest when the nest is placed within the tub 12b and on the ledge 50 of the sidewalls 40. However, the support columns of FIGS. 6 and 7 are elongated in the horizontal direction, so as to present a larger top surface that supports the bottom-most nest, with a top surface of the support columns sized so as to extend over half a length of the nest. Thus, each of the support columns may be sized so as to make contact with the bottom ends of between five (5) and ten (1)0 chimneys 32 of the nest 14, as a non-limiting example.

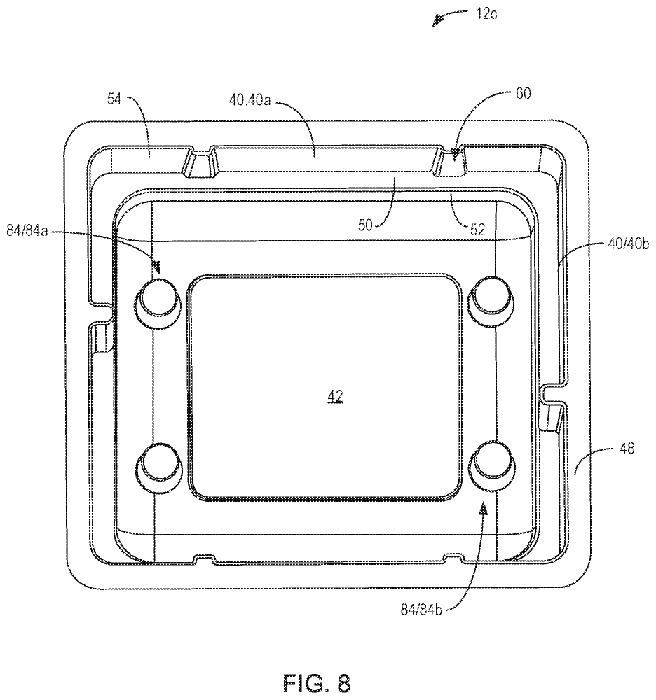

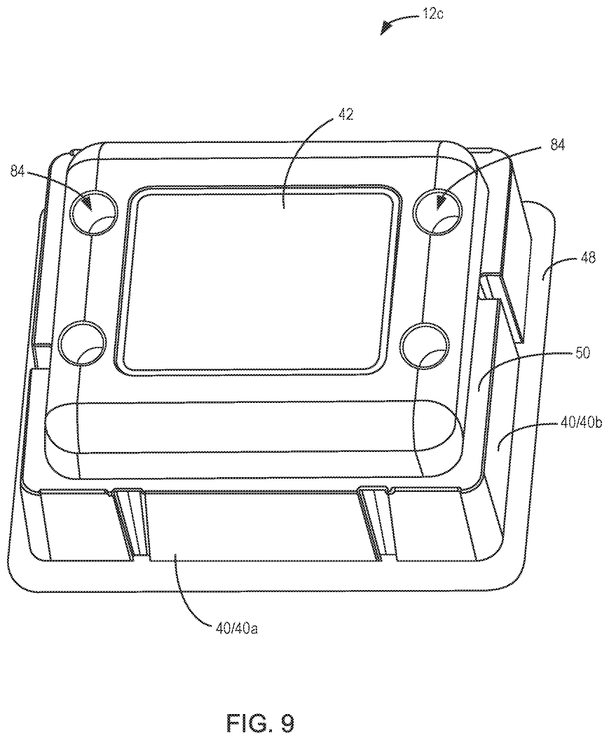

Referring to FIGS. 8 and 9, a tub 12c is shown that includes support members provided as a plurality of cylindrical support columns 84 spaced apart on the bottom wall 42. That is, a first pair of cylindrical support columns 84a is provided that is generally positioned so as to be spaced apart from one sidewall (of the second opposed lateral sidewalls 40b) and a second pair of cylindrical support columns 84b is provided that is generally positioned so as to be spaced apart from an opposite sidewall (of the second opposed lateral sidewalls 40b). Each of the cylindrical support columns 84 extends upwardly from the bottom wall 42 to a flat top surface 86 and has a height that positions the top surface 86 vertically below the ledges 50 formed on sidewalls 40, such that the top surfaces 86 of the cylindrical support columns 84 are positioned so as to engage a bottom edge 72 of a plurality of chimneys 32 of the bottom-most nest 14 (FIG. 2) when the nest is placed within the tub 12c and on the ledge 50 of the sidewalls 40. In some embodiments, the cylindrical support columns 84 may be positioned such that each support column 84 is centered between a plurality of adjacent chimneys 32 of the nest 14. Thus, for example, each cylindrical support column 84 may be centered between a grouping of four (4) adjacent chimneys 32 (when the chimneys 32 are in a linear array on nest 14) or may be centered between a grouping of three (3) adjacent chimneys 32 (when the chimneys 32 are in a hexagonal array on nest 14).

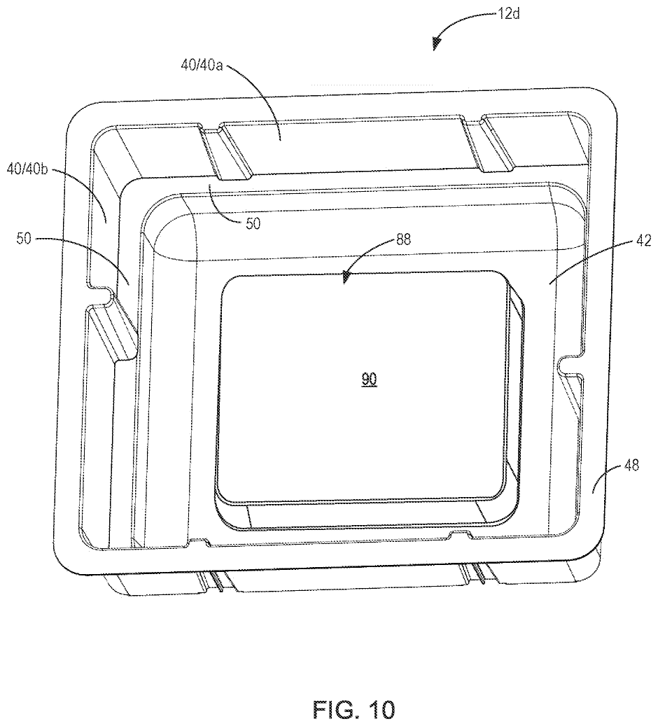

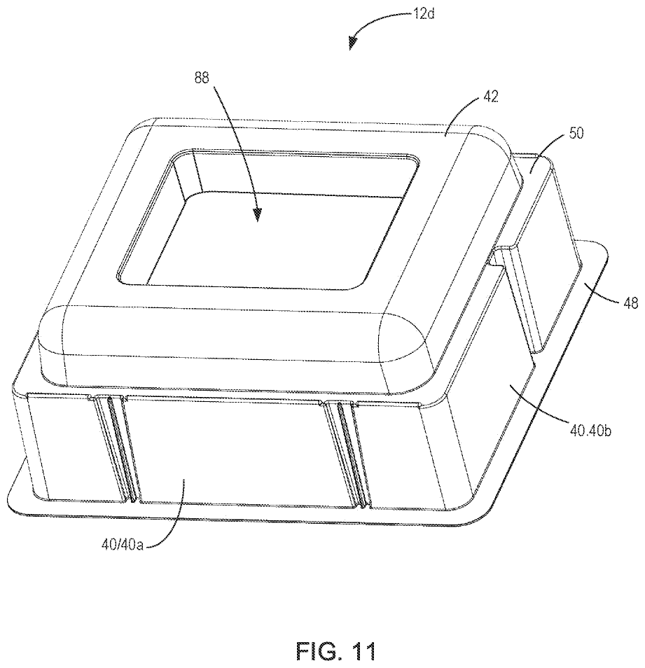

Referring to FIGS. 10 and 11, a tub 12d is shown that includes a support member provided as a single central support column 88 having a generally box-shaped profile. The central support column 88 extends upwardly from the bottom wall 42 to a flat top surface 90, with a height of the central support column 88 positioning the top surface 90 vertically below the ledges 50 formed on sidewalls 40, such that the top surface 90 of the central support column 88 is positioned so as to engage a bottom edge 72 of a plurality of chimneys 32 of the bottom-most nest 14 (FIG. 2) when the nest is placed within the tub 12d and on the ledge 50 of the sidewalls 40. In some embodiments, the central support column 88 may be sized so as to make contact with the bottom edge 72 of a majority of the chimneys 32 of nest 14, including all chimneys 32 located in a central region of the nest 14 that would be provided the least support from engagement between the ledges 50 and the edges of support plate 20 of nest 14.

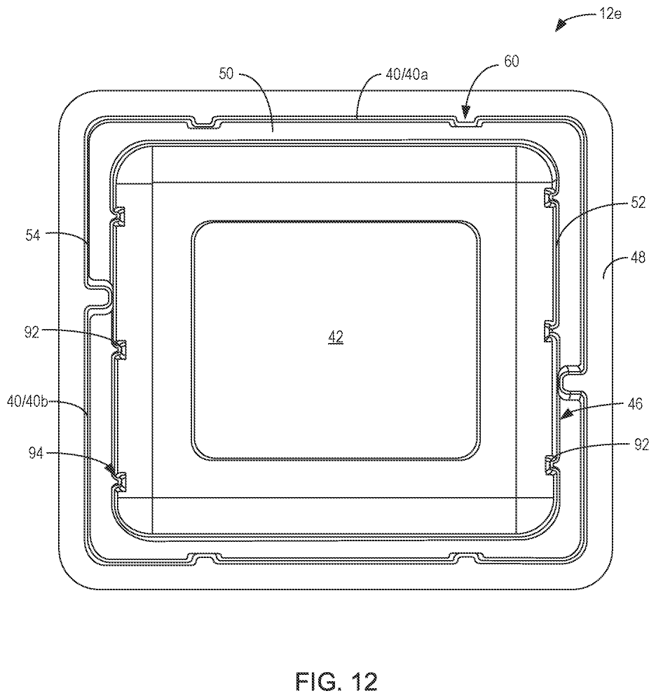

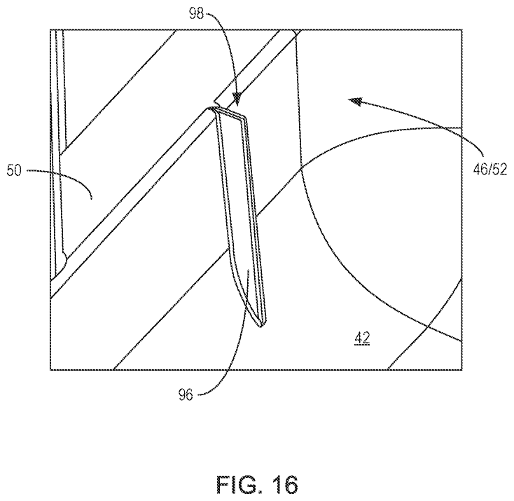

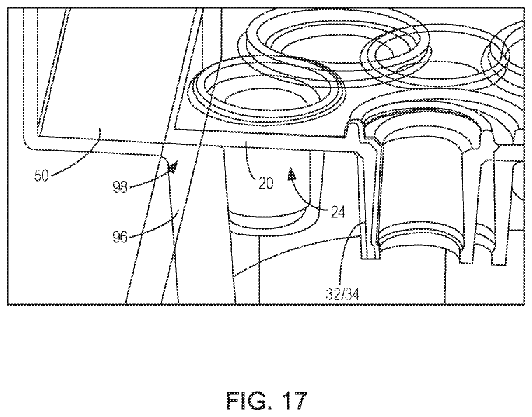

Referring now to FIGS. 12-17, and with continued reference to FIG. 2, additional tub embodiments are provided that include support members that may engage the lower surface 24 of the support plate 20 of the bottom-most nest 14. The additional support provided to the bottom-most nest 14 by the support members may prevent a downward deflection or bending of the nest 14 that might occur if the nest is not properly supported in the tub 12.

Referring first to FIGS. 12-14, a tub 12e is shown that includes support members provided as a plurality of steps 92 that are formed as part of the lower sidewall portion 52 on at least some of the sidewalls 40. In some embodiments, an arrangement of three (3) steps 92 is provided on the lower sidewall portion 52 of a first sidewall (of the second opposed lateral sidewalls 40b) and an arrangement of three (3) steps 92 is provided on the lower sidewall portion 52 of a second sidewall (of the second opposed lateral sidewalls 40b) that is opposite the first sidewall—where the transverse sides 30 of nest(s) 14 are positioned when placed within tub 12a and with it recognized that the ledges 50 provide less support to a bottom-most nest 14 along the transverse sides 30.

Each of the plurality of steps 92 may have a structure somewhat similar to that of the projections 62 of guide features—with the steps 92 having a vertically elongated structure. The steps 92 extend inwardly from the interior surface 46 of the lower sidewall portion 52, so as to extend out into a volume defined by the lower wall portions 52 and bottom wall 42. The steps 92 extend up vertically from the bottom wall 42 to a top surface 94 that is positioned at a same height as the ledges 50—i.e., the top surface 94 of steps 92 is vertically aligned with ledges 50. With the steps 92 configured as such, the top surface 94 of steps 92 are positioned so as to engage a lower surface 24 of a support plate 20 of the bottom-most nest 14 when the nest is placed within the tub 12e and on the ledges 50 of the sidewalls 40. The top surface 94 of each of the steps 92 is dimensioned such that it may fit between adjacent chimneys 32 of the nest 14, to make contact with the lower surface 24 of the support plate 20 of the bottom-most nest 14.

With the ledges 50 of tub 12e providing some support to the lower surface 24 of support plate 20 of the bottom-most nest 14 and the steps 92 providing additional support to the lower surface 24 of the support plate 20 of the bottom-most nest 14, the nest is provided with multiple points/types of support within the tub 12e. With this support, the bottom-most nest 14 is prevented from deflecting/bending downward by an undesired amount, even when the bottom-most nest 14 is loaded with stoppers and additional nests are loaded on top of the bottom-most nest 14.

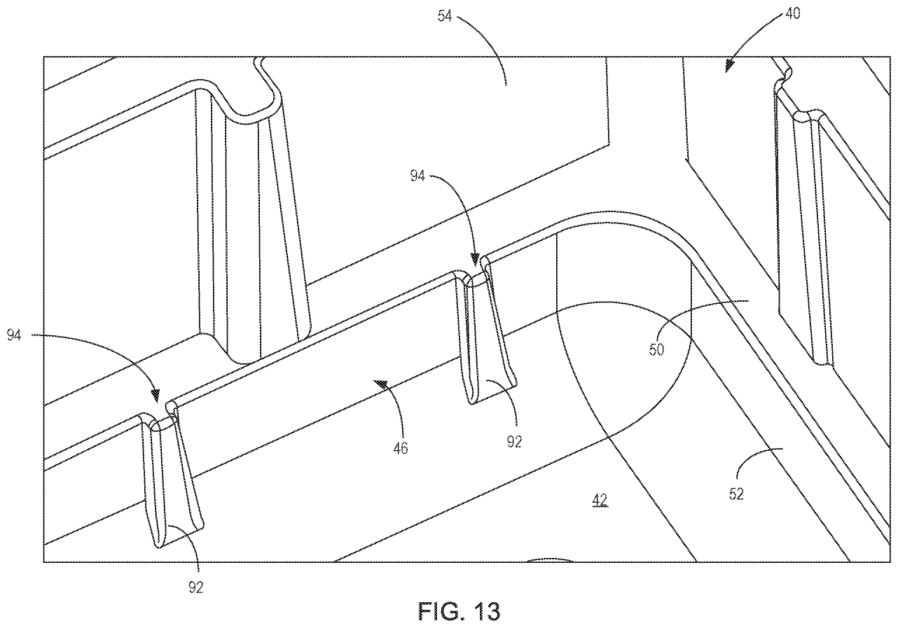

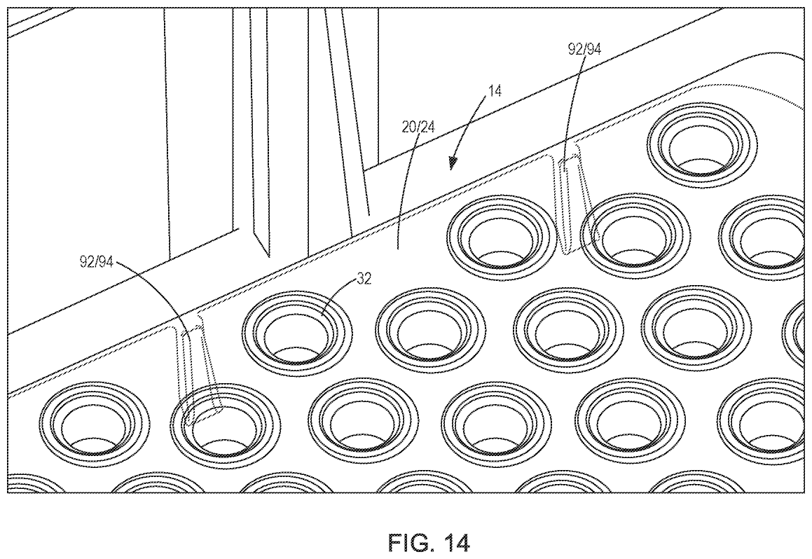

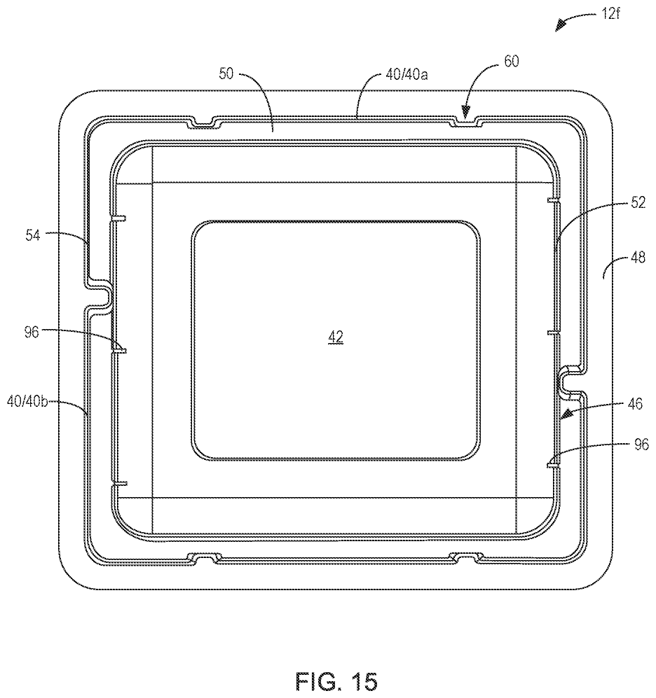

Referring now to FIGS. 15-17, a tub 12f is shown that includes support members provided as a plurality of ribs 96 that are formed as part of the lower sidewall portion 52 on at least some of the sidewalls 40. Similar to the steps 92 provided on tub 12e in the embodiment of FIGS. 12 and 13, an arrangement of three (3) ribs 96 may be provided on the lower sidewall portion 52 of a first sidewall (of the second opposed lateral sidewalls 40b) and an arrangement of three (3) ribs 96 may be provided on the lower sidewall portion 52 of a second sidewall (of the second opposed lateral sidewalls 40b) that is opposite the first sidewall.

Each of the ribs 96 may have a structure somewhat similar to that of the steps 92 provided on tub in the embodiment of FIGS. 12-14, except that the ribs 96 have a thinner/narrower profile as compared to the steps 92. The ribs 96 extend inwardly from the interior surface 46 of the lower sidewall portion 52, so as to extend out into a volume defined by the lower wall portions 52 and bottom wall 42. The ribs 96 extend up vertically from the bottom wall 42 to a top surface 98 that is positioned at a same height as the ledges 50—i.e., the top surface 98 of ribs 96 is vertically aligned with ledges 50. With the ribs 96 configured as such, the top surface 98 of ribs 86 are positioned so as to engage a lower surface 24 of a support plate 20 of the bottom-most nest 14 when the nest is placed within the tub 12f and on the ledges 50 of the sidewalls 40. The top surface 9 of each of the ribs 96 is dimensioned such that it may fit between adjacent chimneys 32 of the nest 14, to make contact with the lower surface 24 of the support plate 20 of the bottom-most nest 14 and provide support thereto. With this support provided by ribs 96, the bottom-most nest 14 is prevented from deflecting/bending downward by an undesired amount, even when the bottom-most nest 14 is loaded with stoppers and additional nests are loaded on top of the bottom-most nest 14.

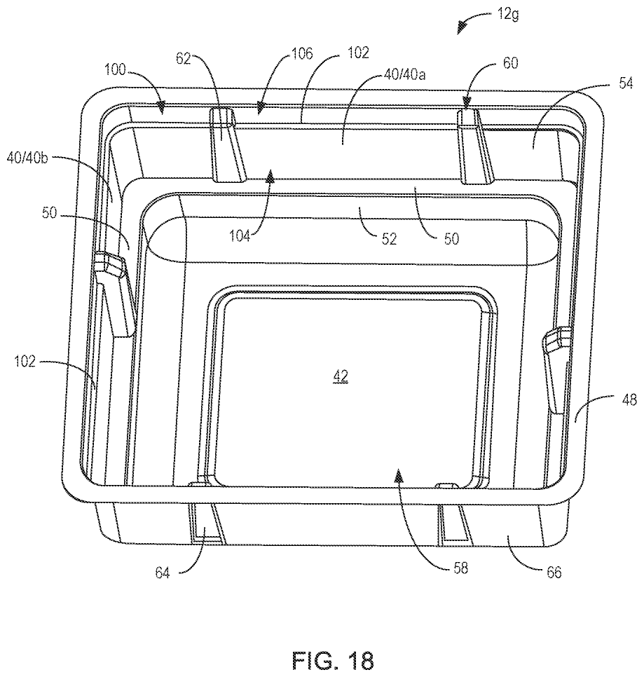

Referring now to FIGS. 18-27, according to additional aspects of the disclosure, tubs 12 may be provided that include features thereon that make the sidewalls 40 thereof more resistant to deformation or deflection that might occur after prolonged periods of use. That is, the tubs 12 include features thereon that make the sidewalls 40 thereof more resistant to a “box effect” type inward bowing or deflection that might result from the sidewalls 40 undergoing stress relaxation—with it recognized that injection molded tubs can experience such stress relaxation as the tubs age. As described in further detail below, various features may be included on a tub 12 to resist such inward bowing or deflection of the sidewalls 40.

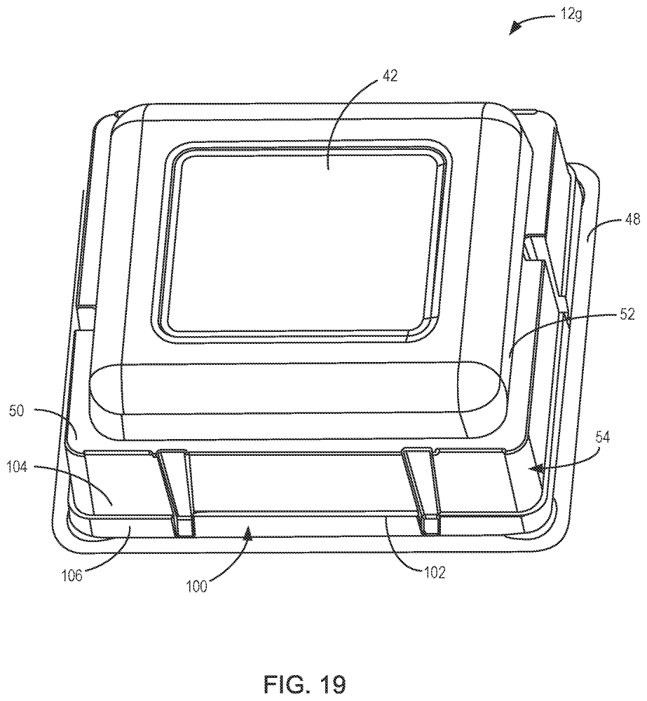

Referring first to FIGS. 18 and 19, a tub 12g is shown according to another embodiment of the disclosure. The structure of tub 12g is substantially similar to the structure of tubs 12a-12f, and thus common features between the tubs are numbered identically. In general, tub 12g includes sidewalls 40, bottom wall 42, and top flange 48—with sidewalls 40 having a stepped construction that defines a ledge 50 positioned between a lower sidewall portion 52 and an upper sidewall portion 54 of each sidewall 40. The tub 12g also includes guide features 60 thereon that provide for alignment and engaging of multiple tubs when it is desired to stack tubs together. In the illustrated embodiment, both first opposed lateral sidewalls 40a and second opposed lateral sidewalls 40b are shown as including guide features 60—with the first opposed lateral sidewalls 40a including two guide features 60 thereon and the second opposed lateral sidewalls 40b including a single guide feature thereon. As described above, the guide features 60 are formed so as to appear as projections 62 that extend inwardly from the interior surface 46 of the sidewalls 40 and appear as recesses 64 depressed inwardly into an exterior surface 66 of the sidewalls 40.

According to aspects of the disclosure, the tub 12g also includes thereon a stiffening belt 100 formed on the interior surface 46 of sidewalls 40—on the upper sidewall portion 54 thereof of each sidewall 40. In the illustrated embodiment, the stiffening belt 100 is provided as a shelf or ledge 102 (“stiffening ledge”) that is formed on the upper sidewall portion 54 of each sidewall 40. That is, the upper sidewall portion 54 may be formed to have a stepped construction—with the upper sidewall portion 54 having a lower first portion 104 and an upper second portion 106 that is separated by the stiffening ledge 102. The stiffening ledge 102 may have a horizontal (or generally horizontal) orientation, with the ledge 102 extending outwardly from an upper edge of the second portion 106 to a lower edge of the first wall portion 104 of upper sidewall portion 54. The stiffening ledge 102 may have a constant and generally narrow width, such as a width of a few millimeters, that does not greater increase the size of the opening 58 to tub 12g, but is sufficient to increase the rigidity of the upper sidewall portion 54 of sidewalls 40. With the increased rigidity provided by stiffening ledge 102, the sidewalls 40 are made more resistant to an inward bowing that might be caused by a stress relaxation of the injection molded tub 12g.

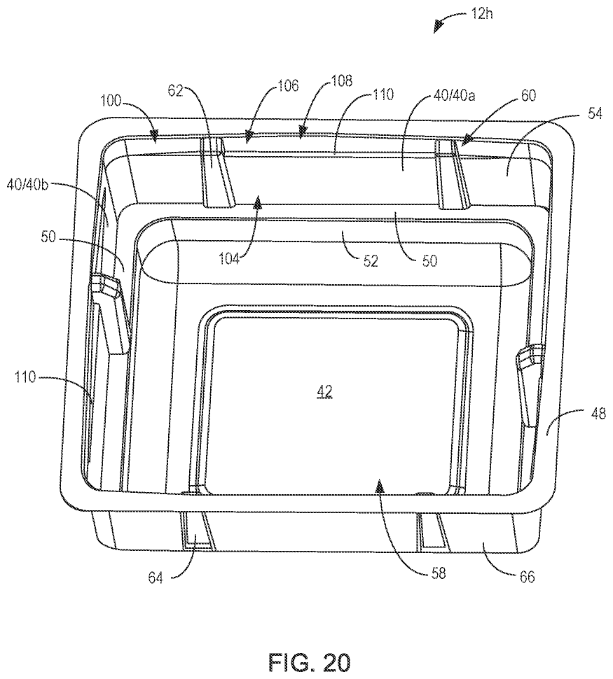

Referring now to FIGS. 20 and 21, a tub 12h is shown according to another embodiment of the disclosure—with the tub 12h again including a stiffening belt 108 formed on the interior surface 46 of sidewalls 40. The stiffening belt 108 is again provided as stiffening ledge 110 that is formed on the upper sidewall portion 54 of each sidewall 40. However, as compared to the stiffening ledge 102 provided on tub 12h that has a constant width, the stiffening ledge 110 in tub 12h is formed as an “evolutive ledge” that has a variable width along a length of each sidewall 40. As a non-limiting example, for the stiffening ledge 110 provided on each first opposed lateral sidewalls 40a, the stiffening ledge 110 has an increased width along a length of the sidewall 40a that is between the guide features 60 provided thereon, with a thickest width of the stiffening ledge 110 provided at a (horizontal) mid-point of the sidewall 40a. The width of the stiffening ledge 110 tapers down as it progresses to the corners of tub 12h on each end of the sidewall 40. The evolutive stiffening ledge 110 again functions to increase the rigidity of the sidewalls 40, such that the sidewalls 40 are made more resistant to an inward bowing that might be caused by a stress relaxation of the injection molded tub 12h.

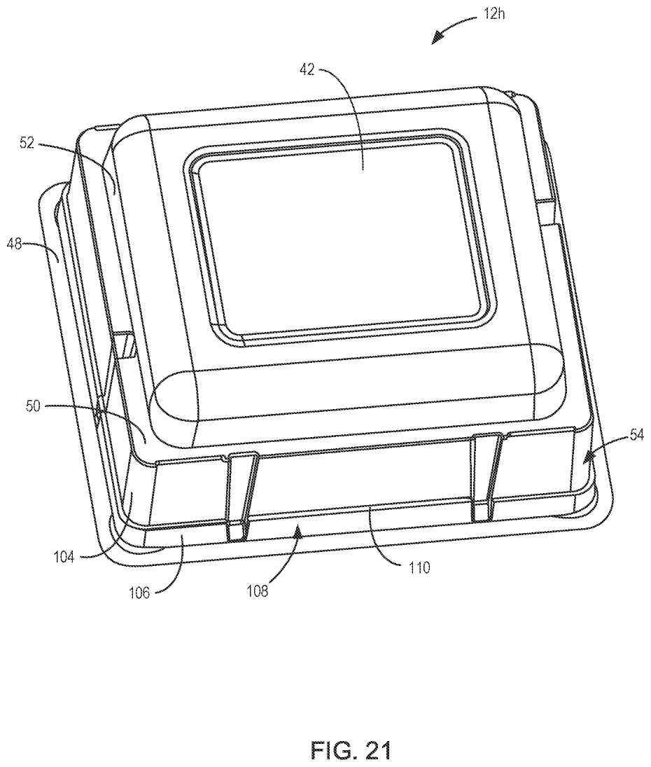

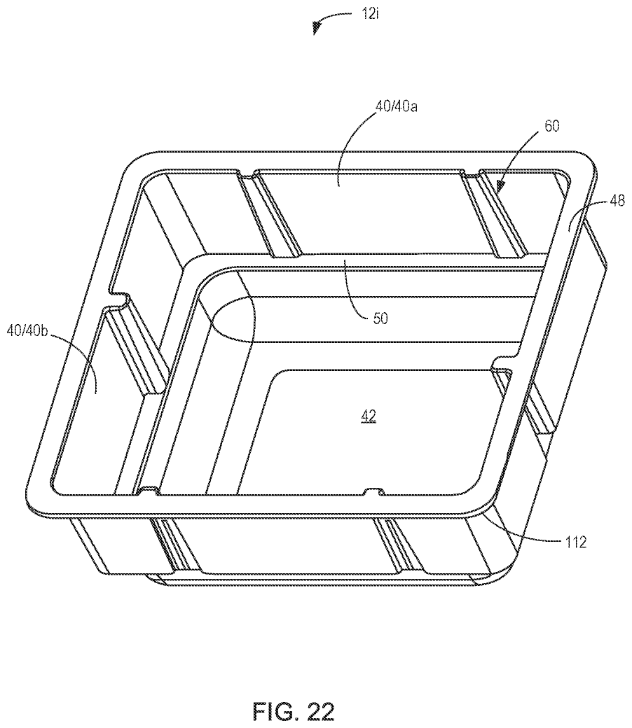

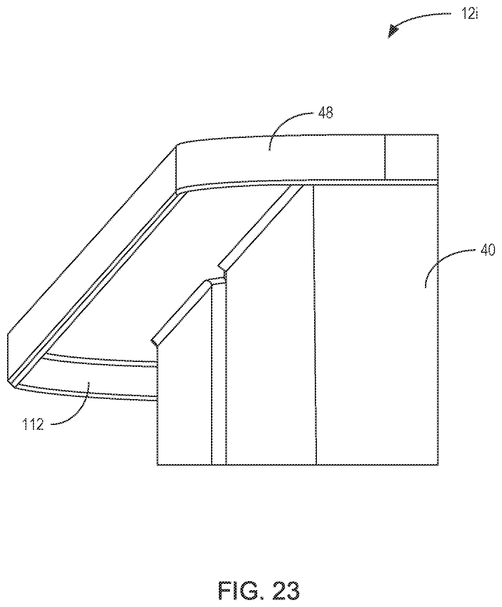

Referring now to FIGS. 22 and 23, a tub 12i is shown according to another embodiment of the disclosure. In order to make the sidewalls 40 thereof more resistant to deformation or deflection, the top flange 48 of tub 12i is structured to increase the rigidity of the sidewalls 40. That is, top flange 48 includes a vertically oriented lip 112 that formed is about the outer edge thereof, with the lip 112 extending downwardly from a horizontal top surface of the flange, back toward the bottom wall 42. The lip 112 is formed about an entire perimeter of the top flange 48, such that the lip 112 is provided as a single, continuous structure. The vertically oriented lip 112 formed on top flange 48 functions to increase a rigidity of the plurality of sidewalls 40 and make them more resistant to an inward bowing thereof that might be caused by a stress relaxation of the injection molded tub 12i.

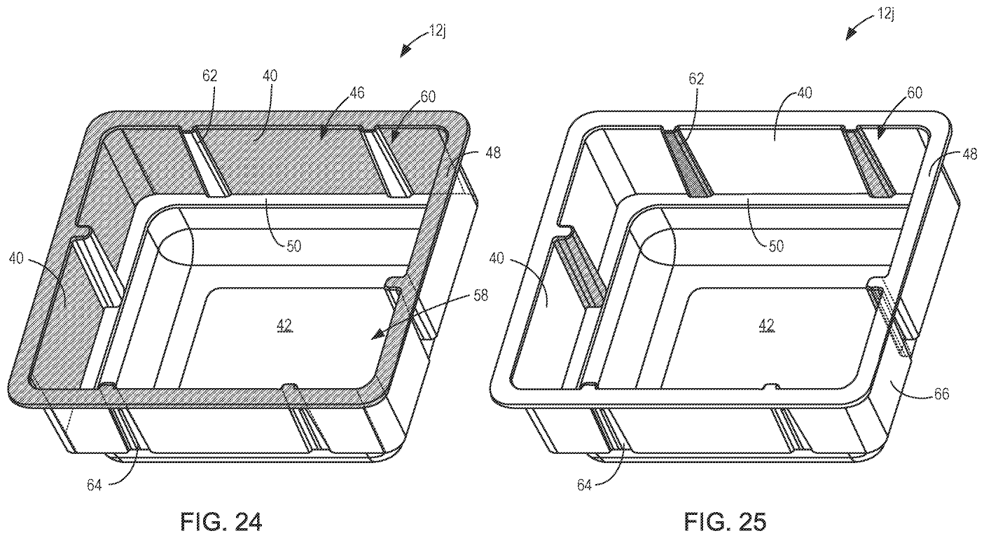

Referring now to FIGS. 24-27, tubs 12j, 12k are shown according to additional embodiments of the disclosure. The tubs 12j, 12k—rather than including a distinct feature that makes the sidewalls 40 thereof more resistant to deformation or deflection, as is the case with tubs 12h, 12i—are structured to so as to inherently provide increased resistance to deformation or deflection of the sidewalls 40. That is, the sidewalls 40 of tubs 12h, 12i are formed to have an increased thickness that makes the sidewalls 40 more resistant to deformation or deflection.

Referring first to FIGS. 24 and 25, tub 12j is formed such that the sidewalls 40 thereof have an increased thickness as compared to tubs 12a-12i, with the sidewalls 40 also formed to have a thickness that is greater than a thickness of the bottom wall 42. In some embodiments, the sidewalls 40 may be increased in thickness by 1.0 to 1.5 mm, as a non-limiting example. In some embodiments, and as shown in FIG. 24, the additional material added to sidewalls 40 (to increase the thickness thereof) may be added to the interior surface 46 of the sidewalls 40 (thereby reducing dimensions of the opening 58 of tub 12j). In other embodiments, and as shown in FIG. 25, the additional material added to sidewalls 40 (to increase the thickness thereof) may be added to the exterior surface 66 of the sidewalls 40.

According to aspects of the disclosure, the thickness of the sidewalls 40 may be increased for the entirety of the sidewalls 40 or may be increased at all locations except for where guide features 60 are present. In some embodiments, the thickness of top flange 48 may be increased in connection with the thickness of sidewalls 40. The thickness of the top flange 48 may be increased by the same amount as the thickness of sidewalls 40. With the increased thickness of the sidewalls 40 (and top flange 48), the sidewalls 40 of tub 12j may be made more resistant to an inward bowing thereof that might be caused by a stress relaxation of the injection molded tub 12j.

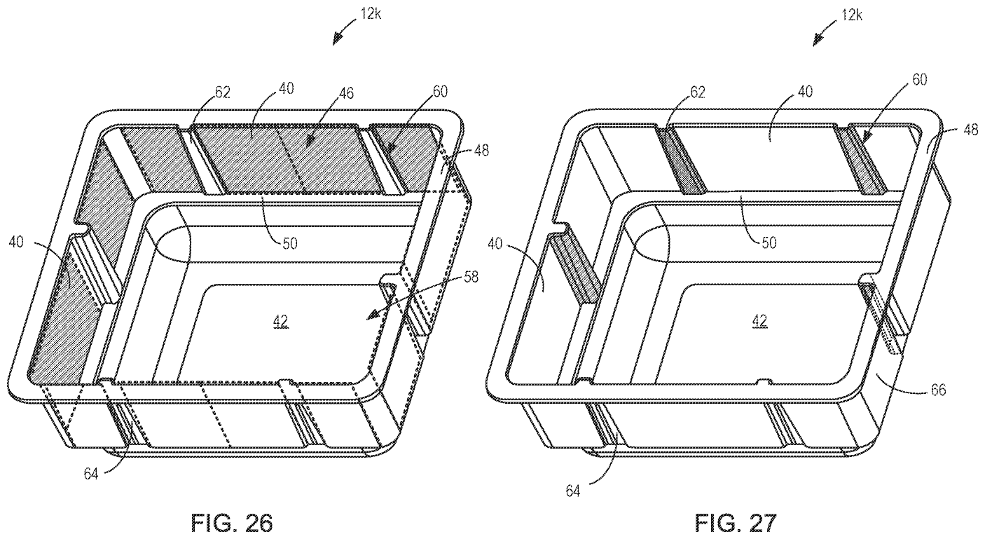

Referring now to FIGS. 26 and 27, a tub 12k is provided where the sidewalls 40 are formed to have a variable increased thickness that makes the sidewalls 40 more resistant to deformation or deflection. That is, as compared to the sidewalls 40 of tub 12j that were increased in thickness along an entirety thereof (except, optionally, for guide features 60), the sidewalls 40 of tub 12k are formed to have an “evolutive thickness” that may vary. As one example, the thickness of each sidewall 40 may be increased by the greatest amount at/near the horizontal midpoint thereof, with the thickness then tapering down as the sidewall 48 extends toward a corner of the tub 12k. In some embodiments, and as shown in FIG. 26, the additional material added to sidewalls 40 (to increase the thickness thereof) may be added to the interior surface 46 of the sidewalls 40 (thereby reducing dimensions of the opening 58 of tub 12k). In other embodiments, and as shown in FIG. 27, the additional material added to sidewalls 40 (to increase the thickness thereof) may be added to the exterior surface 66 of the sidewalls 40. With the increased thickness of the sidewalls 40—with a larger increase of thickness at desired locations—the sidewalls 40 of tub 12k may be made more resistant to an inward bowing thereof that might be caused by a stress relaxation of the injection molded tub 12k.

Beneficially, embodiments of the invention thus provide tubs that are used for transporting a plurality of nests and the medical components (e.g., stoppers) held thereby. The tubs are designed so as to provide additional support for the nests retained therein, so as to prevent unwanted deflection of the nests. The tubs are further designed to have increased resistance to sidewall deflection and deformation that might result from stress relaxation that occurs in the sidewalls after prolonged periods of time, so as to prevent potential issues with removing the nests from the tub that might result from such deflection and deformation.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A tub for holding one or more nests each including a plurality of chimneys configured to retain medical components therein, the tub comprising:
a bottom wall;
a plurality of sidewalls extending upwardly from the bottom wall and including an inner wall surface and an outer wall surface, wherein each of the plurality of sidewalls comprises a stepped wall including a lower wall portion and an upper wall portion, with a ledge formed between the lower wall portion and the upper wall portion, the ledge comprising a horizontal ledge in parallel with the bottom wall;
a top flange formed about a top edge of the plurality of sidewalls; and
one or more support members extending up from the bottom wall, each of the one or more support members having a top surface that is vertically aligned with or vertically below the ledges of the plurality of sidewalls, with the one or more support members positioned to support a bottommost nest of the one or more nests when placed within the tub.

2. The tub of claim 1, wherein the one or more support members comprise a plurality of steps formed as part of the plurality of sidewalls, each of the plurality of steps extending inwardly from a respective lower wall portion and configured to fit between adjacent chimneys of the bottommost nest, with the top surface of the each of the plurality of steps vertically aligned with the ledges of the plurality of sidewalls so as to contact a bottom surface of a support plate of the bottommost nest when placed within the tub.

3. The tub of claim 1, wherein the one or more support members comprise a plurality of ribs formed as part of the plurality of sidewalls, each of the plurality of ribs extending inwardly from a respective lower wall portion and configured to fit between adjacent chimneys of the bottommost nest, with the top surface of the each of the plurality of ribs vertically aligned with the ledges of the plurality of sidewalls so as to contact a bottom surface of a support plate of the bottommost nest when placed within the tub.

4. The tub of claim 1, wherein the one or more support members comprise one or more support columns extending up from the bottom wall, with the top surface of each of the one or more support columns positioned vertically below the ledges of the plurality of sidewalls, so as to support a lower edge of the plurality of chimneys of the bottommost nest when placed within the tub.

5. The tub of claim 4, wherein the one or more support columns comprise:
a first support column positioned adjacent a first sidewall of the plurality of sidewalls; and
a second support column positioned adjacent a second sidewall of the plurality of sidewalls opposite the first sidewall.

6. The tub of claim 4, wherein the one or more support columns comprise a single support column positioned so as to be centered on the bottom wall, the single support column covering a majority of the bottom wall.

7. The tub of claim 4, wherein the one or more support columns comprise:
a first pair of cylindrical support columns spaced apart from a first sidewall of the plurality of sidewalls; and
a second pair of cylindrical support columns spaced apart from a second sidewall of the plurality of sidewalls opposite the first sidewall.

8. The tub of claim 1, wherein the ledge extends outwardly from a top edge of the lower wall portion to a bottom edge of the upper wall portion, and wherein the ledge is configured to support a support plate of the bottom-most nest of the one or more nests held in the tub.

9. The tub of claim 8, wherein each of the plurality of sidewalls includes one or more guide features thereon extending upwardly from the ledge, with each of the one or more guide features defining a projection on the inner wall surface and a recess on the outer wall surface.

10. A packaging system for storing a plurality of medical components, the packaging system comprising:
one or more nests each configured to retain a plurality of medical components, each of the one or more nests comprising:
a support plate having a top surface and a bottom surface; and
a plurality of chimneys formed on the support plate and configured to retain the plurality of medical components therein, each of the plurality of chimneys defining an opening in the support plate and comprising a cylindrical wall extending down from the bottom surface of the support plate, the cylindrical wall having a top edge joined to the support plate and a bottom edge;
a tub comprising:
a bottom wall;
a plurality of sidewalls extending upwardly from the bottom wall and including an inner wall surface and an outer wall surface, wherein each of the plurality of sidewalls comprises a stepped wall including a lower wall portion and an upper wall portion, with a ledge formed between the lower wall portion and the upper wall portion, the ledge comprising a horizontal ledge in parallel with the bottom wall;
a top flange formed about a top edge of the plurality of sidewalls; and
one or more support members extending up from the bottom wall, each of the one or more support members having a top surface that is vertically aligned with or vertically below the ledges of the plurality of sidewalls, with the one or more support members positioned to support a bottommost nest of the one or more nests when placed within the tub; and
a sealing cover applied over the tub and joined to the top flange.

11. The packaging system of claim 10, wherein the top surface of each of the one or more support members is vertically aligned with the ledges of the plurality of sidewalls, with the one or more support members configured to fit between adjacent chimneys of the bottommost nest and contact the bottom surface of the support plate of the bottommost nest when placed within the tub.

12. The packaging system of claim 10, wherein the top surface of each of the one or more support members is positioned vertically below the ledges of the plurality of sidewalls, with the one or more support members configured to contact the bottom edges of the plurality of chimneys of the bottommost nest when placed within the tub.

13. A tub for holding one or more nests each configured to retain a plurality of medical components, the tub comprising:
a bottom wall;
a plurality of sidewalls extending upwardly from the bottom wall and including an inner wall surface and an outer wall surface, wherein each of the plurality of sidewalls comprises a stepped wall including a lower wall portion and an upper wall portion, with a horizontal oriented nest ledge formed between the lower wall portion and the upper wall portion, the nest ledge configured to support a bottom-most nest of the one or more nests; and
a top flange formed about a top edge of the plurality of sidewalls, with the top flange defining an upper opening of the tub;
wherein the upper wall portion on each of the plurality of sidewalls comprises a stiffening belt formed on the inner wall surface thereof, the stiffening belt configured to resist an inward bowing of the respective sidewall.

14. The tub of claim 12, wherein the upper wall portion comprises a stepped wall including a first portion and a second portion, and wherein the stiffening belt comprises a stiffening ledge formed between the first portion and the second portion.

15. The tub of claim 14, wherein the stiffening ledge on each respective sidewall of the plurality of sidewalls comprises a tapered ledge having a variable width along a length of the respective sidewall.

16. The tub of claim 13, wherein each of the plurality of sidewalls includes one or more guide features thereon extending upwardly from the nest ledge, with each of the one or more guide features defining a projection on the inner wall surface and a recess on the outer wall surface, and wherein the one or more guide features on each first of the plurality of sidewalls intersects the stiffening belt formed thereon.

17. A tub for holding one or more nests each configured to retain a plurality of medical components, the tub comprising:
a bottom wall;
a plurality of sidewalls extending upwardly from the bottom wall and including an inner wall surface and an outer wall surface, wherein each of the plurality of sidewalls comprises a stepped wall including a lower wall portion and an upper wall portion, with a horizontal oriented nest ledge formed between the lower wall portion and the upper wall portion, the nest ledge configured to support a bottom-most nest of the one or more nests; and
a top flange formed about a top edge of the plurality of sidewalls, with the top flange defining an upper opening of the tub;
wherein the top flange comprises a vertically oriented lip formed about the outer edge thereof and extending downwardly therefrom, back toward the bottom wall, wherein the top flange and vertically oriented lip increase a rigidity of the plurality of sidewalls, such that the plurality of sidewalls are resistant to an inward bowing thereof.

18. A tub for holding one or more nests each configured to retain a plurality of medical components, the tub comprising:
a bottom wall;
a plurality of sidewalls extending upwardly from the bottom wall and including an inner wall surface and an outer wall surface, wherein each of the plurality of sidewalls comprises a stepped wall including a lower wall portion and an upper wall portion, with a horizontal oriented nest ledge formed between the lower wall portion and the upper wall portion, the nest ledge configured to support a bottom-most nest of the one or more nests; and
a top flange formed about a top edge of the plurality of sidewalls, with the top flange defining an upper opening of the tub;
wherein a thickness of each of the plurality of sidewalls is greater than a thickness of the bottom wall, such that the plurality of sidewalls resist an inward bowing.

19. The tub of claim 18, wherein each of the plurality of sidewalls includes one or more guide features thereon extending upwardly from the nest ledge, with each of the one or more guide features defining a projection on the inner wall surface and a recess on the outer wall surface; and
wherein the thickness of each of the plurality of sidewalls is increased at locations of the projections of the guide features, at locations other than the projections of the guide features, or at both the locations of the projections of the guide features and locations other than the projections of the guide features, such that the plurality of sidewalls may have a varied thickness or a constant thickness.

20. The tub of claim 19, wherein a thickness of the top flange is greater than a thickness of the bottom wall and equal to the thickness of each of the plurality of sidewalls.
